(19) United States
(12) Patent Application Publication
(10) Pub. No.: US 2020/0109099 A1
(43) Pub. Date: Apr. 9, 2020

(54) POLYPHENYLMETHANOL SURFACTANTS

(71) Applicant: Huntsman Petrochemical LLC, The Woodlands, TX (US)

(72) Inventors: Matthew T. Meredith, The Woodlands, TX (US); Alan J. Stern, The Woodlands, TX (US)

(21) Appl. No.: 16/611,714

(22) PCT Filed: May 29, 2018
(86) PCT No.: PCT/US2018/034844

§ 371 Date: Nov. 7, 2019

Related U.S. Application Data
(60) Provisional application No. 62/516,747, filed on Jun. 8, 2017.

(51) Int. Cl. C07C 43/178; A61K 8/39; A01N 25/30; C07C 43/164; C07C 305/10; C07F 9/09; A61K 8/46; A61K 8/55

(52) U.S. Cl. CPC C07C 43/1785; A61K 8/39; A01N 25/30; C07C 43/164; A61K 2800/10; C07F 9/091; A61K 8/463; A61K 8/556; C07C 2601/16; C07C 305/10

(57) ABSTRACT

The present disclosure provides a composition comprising a surfactant compound containing aromatic groups in the hydrophobe. Also disclosed are personal care formulations and performance chemical formulations, such as agrochemical formulations, that comprise such surfactant compounds.

### CROSS REFERENCE TO RELATED APPLICATIONS

The present application claims priority to U.S. Provisional Patent Application Ser. No. 62/516,747, filed Jun. 8, 2017, the entire contents of which is hereby expressly incorporated herein by reference.

### STATEMENT REGARDING FEDERALLY SPONSORED RESEARCH OR DEVELOPMENT

Not applicable.

### FIELD OF THE INVENTION

The present disclosure generally relates to surfactant compounds derived from diphenylmethanol and triphenylmethanol, each of which may be optionally alkylated, and methods for preparing such compounds. The surfactant compounds may be used in a variety of applications including, but not limited to, cleaning, fabric treatment, hair conditioning, agricultural, personal care and antimicrobial formulations, metal-working fluids, polyurethane foam and oil field formulations.

### BACKGROUND

Surfactants containing aromatic groups in the hydrophobe are important in many different applications (agrochemicals, detergents, cleaning, personal care, enhanced oil recovery, etc.) due to their unique functionality, high performance and low cost. Alkyl phenol alkoxylates (hereinafter “APAs”) are one class of such surfactants which have achieved wide acceptance in the art. APAs can be easily tailored for specific applications by selecting certain alkyl substituents on the aromatic group and controlling the number of repeating alkoxy components attached to the phenolic oxygen atom. Such customization allows APAs to be employed in a variety of applications including cosmetics, detergents, cleaners, toiletries, oil slick dispersants, deinking surfactants, metal treatment formulations, textile treatments, emulsion formations and emulsion polymerizations.

Another versatile well known class of surfactants containing aromatic groups in the hydrophobe are polyarylphenol alkoxylates, and in particular tristyrylphenol alkoxylates (hereinafter “TSPAs”). Because of their chemical structure, TSPAs are known to possess unique physico-chemical properties making them highly effective for use as emulsifiers, dispersants and stabilizers in various formulations, particularly agrochemical formulations.

Unfortunately, both of these surfactant classes are based on aromatic hydrophobes with undesirable properties such as toxicity and/or environmental persistence problems. For instance, a few studies have suggested that conventional APAs and TSPAs experience delayed or deficient biodegradation, thus generating biodegradation products, such as alkylphenols and tristyrylphenol. Alkylphenols are known to be endocrine disruptors while alkylphenols and tristyrylphenol are known to be persistent environmental pollutants.

The aforementioned concerns have prompted the implementation of governmental and industrial restrictions on the use of APAs and TSPAs in various countries. While various alternatives have been developed, those skilled in the art have since been relatively unsuccessful in identifying any meaningful alternatives to conventional APAs and TSPAs which still contain aromatic groups. Thus, there remains a significant need to identify, develop and employ alternatives to conventional APAs and TSPAs and their precursors which do not possess the characteristics of endocrine disruption or delayed/deficient biodegradation.

### SUMMARY

According to one aspect, there is provided a composition comprising a surfactant compound having the formula (1)

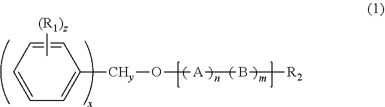

where R₁ is independently hydrogen or a C₁-C₁₈ alkyl group; A is ethyleneoxy, B is a C₃ alkyleneoxy, a C₄ alkyleneoxy or a mixture thereof, where A and B may be randomly distributed, or in the form of two more blocks of any order, R₂ is hydrogen, a C₁-C₁₆ alkyl group, benzyl, diphenylmethyl, triphenylmethyl, SO₃M, CH₂COOM or PO₃M; M is hydrogen, a water-soluble cation, a monovalent metal or a polyvalent metal cation; m is an integer from 0 to 60; n is an integer from 0 to 60; x is 2 or 3; y is 0 or 1; each z is 0, 1 or 2; and wherein m+n=4 to 100 and x+y=3.

In another aspect, there is provided a method of making a composition comprising the surfactant compound of formula (1) by reacting diphenylmethanol (optionally alkylated) or triphenylmethanol (optionally alkylated) with an alkylene oxide or a polyalkylene glycol in the presence of an acid catalyst or a base catalyst and optionally further reaction with an acid moiety and optionally neutralization with a source of alkali metal, alkaline earth metal, amine or ammonia.

In still another aspect, there is provided uses of the composition of the present disclosure, such as in personal care formulations or performance chemical formulations such as agrochemical formulations.

### BRIEF DESCRIPTION OF THE DRAWINGS

FIG. 1 depicts the color scale used in the detergency test;

FIG. 2 depicts the surfactant compounds of the present disclosure capability in reducing the interfacial tension between water and mineral oil;

FIG. 3 depicts the detergency capability of the surfactant compounds of the present disclosure; and

FIG. 4 depicts the hard surface cleaning capability of the surfactant compounds of the present disclosure.

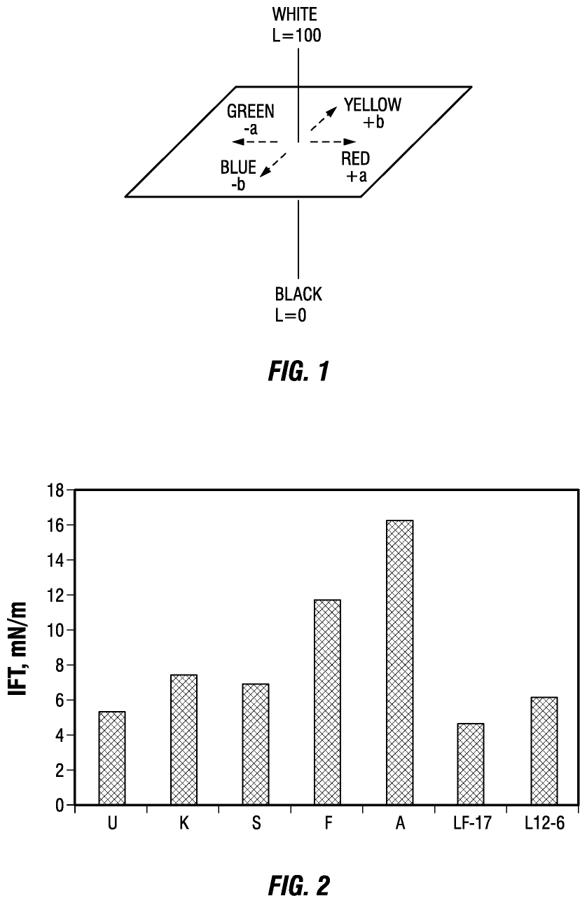

### DETAILED DESCRIPTION

The following terms shall have the following meanings.

If appearing herein, the term “comprising” and derivatives thereof are not intended to exclude the presence of any additional component, step or procedure, whether or not the same is disclosed herein. In order to avoid any doubt, all compositions claimed herein through use of the term “comprising” may include any additional additive, adjuvant, or compound, unless stated to the contrary. In contrast, the term, “consisting essentially of” if appearing herein, excludes from the scope of any succeeding recitation any other component, step or procedure, except those that are not essential to operability and the term “consisting of”, if used, excludes any component, step or procedure not specifically delineated or listed. The term “or”, unless stated otherwise, refers to the listed members individually as well as in any combination.

The articles “a” and “an” are used herein to refer to one or to more than one (i.e. to at least one) of the grammatical objects of the article. By way of example, “an alkylene oxide” means one alkylene oxide or more than one alkylene oxide. The phrases “in one aspect”, “according to one aspect” and the like generally mean the particular feature, structure, or characteristic following the phrase is included in at least one aspect of the present disclosure, and may be included in more than one aspect of the present disclosure. Importantly, such phrases do not necessarily refer to the same aspect. If the specification states a component or feature “may”, “can”, “could”, or “might” be included or have a characteristic, that particular component or feature is not required to be included or have the characteristic.

The term “alkyl” is inclusive of both straight chain and branched chain groups and of cyclic groups. Straight chain and branched chain groups may have up to 20 carbon atoms unless otherwise specified. Cyclic groups can be monocyclic or polycyclic, and in some aspects, can have from 3 to 10 carbon atoms.

The term “substantially free” means, when used with reference to the substantial absence of a component in a composition, that such a component is not present, or if at all, as an incidental impurity or by-product. In other words, the component does not affect the properties of the composition.

The term “performance chemicals formulations” refers to non-personal care formulations that serve a broad variety of applications, and include non-limiting formulations such as, adhesives, agrochemical, fertilizer, biocides, coatings, electronics, household-industrial-institutional (HI&I), inks, membranes, metal working, paper, paints, plastics, printing, plasters, oil field, polyurethane, textile and wood-care formulations.

The term “personal care formulation” refers to such illustrative non-limiting formulations as skin, sun, oil, hair, cosmetic, and preservative formulations, including those to alter the color and appearance of the skin. Potential personal care formulations include, but are not limited to, polymers for increased flexibility in styling, durable styling, and increased humidity resistance for hair, skin, and color cosmetics, sun care water-proof/resistance, wear-resistance, and thermal protecting/enhancing formulations.

According to one aspect, the present disclosure provides a composition including a novel surfactant compound containing aromatic groups in the hydrophobe which may allow the surfactant compound to exhibit the unique functionality, high performance and low cost of APAs and TSPAs, but without the toxicity and/or environmental persistence problems associated with these conventional surfactants. In particular, Applicants have surprisingly found that placement of an alkyl spacer between the aromatic moiety and hydroxyl group, in contrast with the direct attachment of the hydroxyl group to the aromatic moiety in conventional APAs and TSPAs, may promote the biodegradation characteristics of the surfactant compounds of the present disclosure. Initially, it must be understood that the surfactant compounds of the present disclosure are not phenols. That is to say, the indirect attachment of the hydroxyl group to the aromatic moiety of the present surfactant compounds eliminates the donation of electrons from the electron-enriched oxygen group to the aromatic ring. Without wishing to be bound by theory, it is believed that this indirect attachment of the hydroxyl group to the aromatic moiety results in the decreased polarization and polarizability of the present surfactant compounds, thereby increasing their biodegradability. However, it should be noted that such indirect attachment still preserves the surfactancy properties of the inventive surfactant compounds so that they are at least comparable, and in some aspects even improved, to those for conventional APAs and TSPAs.

According to one aspect, the surfactant compound is a compound having the general formula (1)

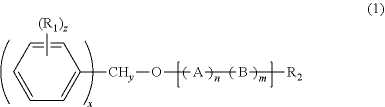

where R₁ is independently hydrogen or a C₁-C₁₈ alkyl group; A is ethyleneoxy, B is a C₃ alkyleneoxy, a C₄ alkyleneoxy or a mixture thereof, where A and B may be randomly distributed, or in the form of two more blocks of any order; R₂ is hydrogen, a C₁-C₁₆ alkyl group, benzyl, diphenylmethyl, triphenylmethyl, SO₃M, CH₂COOM or PO₃M; M is hydrogen, a water-soluble cation, a monovalent metal or a polyvalent metal cation; m is an integer from 0 to 60; n is an integer from 0 to 60; x is 2 or 3; y is 0 or 1; each z is 0, 1 or 2; and wherein m+n=4 to 100 and x+y=3. According to some aspects, in formula (1), B or a block of B units may be positioned closed to the phenyl ring(s) than the A unit(s). According to other aspects, in formula (1), A or a block of A units may be positioned closer to the phenyl ring(s) than the B unit(s).

According to one particular aspect, the surfactant compound is a compound having the formula (1) above and x is 2, y is 1 and each z is 0. In yet another aspect, the surfactant compound is a compound having the formula (1) above and x is 2, y is 1, at least one z is 1 and R₁ is a C₄-C₁₄ alkyl group, while in other aspects R₁ is a C₆-C₁₂ alkyl group or in still other aspects R₁ is a C₈-C₁₀ alkyl group. In still another aspect, the surfactant compound is a compound having the formula (1) above and x is 2, y is 1, at least one z is 2 and at least one R₁ is a C₄-C₁₄ alkyl group, while in other aspects at least one R₁ is a C₆-C₁₂ alkyl group or in still other aspects at least one R₁ is a C₈-C₁₀ alkyl group.

According to one particular aspect, the surfactant compound is a compound having the formula (1) above and R₁ is a C₄-C₆ alkyl group, a C₆-C₈ alkyl group, a C₈-C₁₀ alkyl group, a C₁₀-C₁₂ alkyl group or a C₁₂-C₁₄ alkyl group.

According to another particular aspect, the surfactant compound is a compound having the formula (1) above and x is 3, y is 0 and each z is 0. In yet another aspect, the surfactant compound is a compound having the formula (1) above and x is 3, y is 0, at least one z is 1 and R₁ is a C₄-C₁₄ alkyl group, while in other aspects R₁ is a C₆-C₁₂ alkyl group or in still other aspects R₁ is a C₈-C₁₀ alkyl group. In still another aspect, the surfactant compound is a compound having the formula (1) above and x is 3, y is 0, at least one z is 2 and at least one R₁ is a C₄-C₁₄ alkyl group, or in other aspects at least one R₁ is a C₆-C₁₂ alkyl group or in still other aspects at least one R₁ is a C₈-C₁₀ alkyl group.

According to yet another particular aspect, the surfactant compound is a compound having the formula (1) above and R₂ is hydrogen or methyl. In still another aspect, the surfactant compound is a compound having the formula (1) above and R₂ is diphenylmethyl or triphenylmethyl. In still yet another aspect, the surfactant compound is a compound having the formula (1) above and R₂ is SO₃M or PO₃M. According to one particular aspect, when R₂ is PO₃M, the surfactant compound can include a mono ester, a di ester or a combination thereof.

According to still yet another aspect, the surfactant compound is a compound having the formula (1) above and m is an integer from 0 to 40, n is an integer from 0 to 40 and m+n=6 to 60. In still another aspect, the surfactant compound is a compound having the formula (1) above and m is an integer from 0 to 25, n is an integer from 0 to 25, and m+n=10 to 30. In still another aspect, m+n may equal 8, 10, 12, 14, 15, 16, 18, 20, 22, 24, 25, 26, 28, 30, 32, 34, 35, 36, 38 or 40. In still yet another aspect, the surfactant compound is a compound having the formula (1) and B is chosen such that the m groups are random or block groups of propyleneoxy and butyleneoxy. In yet another aspect, B is propyleneoxy. In still yet another aspect, B is butyleneoxy. In yet another aspect, the surfactant compound is a compound having the formula (1) and A and B, individually, are arranged randomly or in blocks.

According to still yet another aspect, the surfactant compound is a compound having the formula (1) where m is an integer of 4, 5, 6, 8, 10, 12, 14, 15, 16, 18, 20, 22, 24 or 25. According to still yet another aspect, the surfactant compound is a compound having the formula (1) where m is an integer of 26, 28, 30, 32, 34, 35, 36, 38, 40, 42, 44 or 45. According to still yet another aspect, the surfactant compound is a compound having the formula (1) where m is an integer of 46, 48, 50, 52, 54, 55, 56, 58 or 60.

According to still yet another aspect, the surfactant compound is a compound having the formula (1) where n is an integer of 4, 5, 6, 7, 8, 9, 10, 12, 14, 15, 16, 17, 18, 20, 22, 24 or 25. According to still yet another aspect, the surfactant compound is a compound having the formula (1) where n is an integer of 26, 28, 30, 32, 34, 35, 36, 38, 40, 42, 44 or 45. According to still yet another aspect, the surfactant compound is a compound having the formula (1) where n is an integer of 46, 48, 50, 52, 54, 55, 56, 58 or 60.

It has also been found that the inventive surfactant compounds may be prepared by at least two different methods. According to one method, the inventive surfactant compounds may be prepared by direct alkoxylation of diphenylmethanol, triphenylmethanol, or an alkylated derivative of diphenylmethanol or triphenylmethanol using oxides such as ethylene oxide, propylene oxide, butylene oxide and mixtures thereof. Alkylated derivatives of diphenylmethanol and triphenylmethanol may include such species that have one or two C₁-C₁₈ alkyl groups bonded to each phenyl ring of the diphenylmethanol and triphenylmethanol compounds. Alternatively, the inventive surfactant compounds may be prepared by direct condensation of diphenylmethanol, triphenylmethanol and alkylated derivatives of diphenylmethanol or triphenylmethanol with hydroxyl-terminated polyglycol ethers in the presence an acid catalyst. Advantages for this particular method include the avoidance of handling toxic gaseous materials, such as ethylene oxide, and performance of the reaction at atmospheric pressure and relatively low temperatures, such as 50° C.-150° C. or in some aspects 80 C°-120° C.

Thus, in one aspect the surfactant compound of formula (1) is prepared by the polyaddition of alkylene oxide(s) to diphenylmethanol or triphenylmethanol. In another aspect, the surfactant compound of formula (1) is prepared by reacting (condensing) diphenylmethanol or triphenylmethanol with methyl-terminated polyethylene glycol (mPEG), polyethylene glycol (PEG) or other polyalkylene glycol (PAG) in the presence of an acid catalyst. In still other aspects, diphenylmethanol used in the methods above may be obtained from benzophenone which has been hydrogenated in the presence of a hydrogenation catalyst. If desired, the diphenylmethyl alkoxylate or triphenylmethyl alkoxylate that is produced above may be converted to a sulfonate, carboxylate or phosphate by further reaction with an acidic moiety or sodium chloroacetate, and then optionally neutralized with a source of alkali metal, alkaline earth metal, amine or ammonia.

In one particular aspect where the surfactant compound of formula (1) is prepared by alkoxylation, such alkoxylation can be catalyzed by an alkoxylation catalyst including those well known to one skilled in the art. Examples include, but are not limited to, strong bases, such as alkali metal hydroxides and alkaline earth metal hydroxides, Bronsted acids or Lewis acids such as AlCl₃, BF₃, and the like. Catalysts such as hydrotalcite or double metal cyanide (DMC) may be also used when alkoxylates with a narrow distribution are desired.

Additionally, the alkoxylation may be carried out at temperatures in a range of about 80° C.-250° C., such as about 100° C.-220° C. The pressure may be between ambient pressure and 600 bar. If desired, the alkylene oxide may comprise a mixture of inert gas, for example approximately 5% to 60%.

As discussed above, the ethyleneoxy, C₃ alkyleneoxy and C₄ alkyleneoxy units may be arranged within the surfactant compound of formula (1) in any way. Thus, for example, the structural units m and n may be arranged at random or in blocks.

The degree of alkoxylation, i.e. the mean chain length of the polyether chains of aromatic alkoxylates according to the disclosure and their composition (in other words, the values of m and n) can be controlled by the ratio of the molar amounts of diphenylmethanol or triphenylmethanol to alkylene oxide(s) employed in their preparation, and by the reaction conditions. On the one hand, the alkoxylates according to the disclosure comprise at least or more than approximately 4, in some aspects at least or more than approximately 10, in other particular aspects at least or more than approximately 20, in other particular aspects at least or more than approximately 30, in other particular aspects at least or more than approximately 40 and still other aspects at least or more than approximately 50 ethyleneoxy and/or is a C₃ alkyleneoxy and/or C₄ alkyleneoxy units. On the other hand, the alkoxylates according to the disclosure comprise not more than or less than approximately 100, 80, 70 or 60 ethyleneoxy and/or C₃ alkyleneoxy and/or C₄ alkyleneoxy units.

In still another aspect, the composition may further include a solvent. According to one aspect the solvent is water, and in some aspects, de-ionized water. In other aspects a different solvent may be used in addition to or in place of water. Examples of such solvents include, but are not limited to, hydrocarbons (e.g. pentane or hexane), halocarbons (e.g. Freon 113), ethers (e.g. ethylether (Et₂O), tetrahydrofuran (“THF”) or diglyme (diethyleneglycol dimethyl ether)), nitriles (e.g. CH₃CN), or aromatic compounds (e.g. benzotrifluoride). Still further exemplary solvents include lactates, pyruvates, and diols. Solvents can also include, but are not limited to, acetone, 1,4-dioxane, 1,3-dioxolane, ethyl acetate, cyclohexanone, acetone, 1-methyl-2-pyrodidianone (NMP), and methyl ethyl ketone. Other solvents, include dimethylformamide, dimethylacetamide, N-methyl pyrrolidone, ethylene carbonate, propylene carbonate, glycerol and derivatives, naphthalene and substituted versions, acetic acid anhydride, propionic acid and propionic acid anhydride, dimethyl sulfone, benzophenone, diphenyl sulfone, phenol, m-cresol, dimethyl sulfoxide, diphenyl ether, terphenyl, and the like. Still further solvents include propylene glycol propyl ether (PGPE), 3-heptanol, 2-methyl-1-pentanol, 5-methyl-2-hexanol, 3-hexanol, 2-heptanol, 2-hexanol, 2,3-dimethyl-3-pentanol, propylene glycol methyl ether acetate (PGMEA), ethylene glycol, isopropyl alcohol (IPA), n-butyl ether, propylene glycol n-butyl ether (PGBE), 1-butoxy-2-propanol, 2-methyl-3-pentanol, 2-methoxyethyl acetate, 2-butoxyethanol, 2-ethoxyethyl acetoacetate, 1-pentanol, and propylene glycol methyl ether. The solvents enumerated above may be used alone or in combination.

In another aspect, the composition may optionally contain a dispersant. In certain embodiments, the dispersant may be an ionic or a nonionic compound. The ionic or nonionic compound may further comprise a copolymer, an oligomer, or a surfactant other than the surfactant compound of formula (1), alone or in combination. The term copolymer, as used herein, relates to a polymer compound consisting of more than one polymeric compound such as block, star, dendrimer or grafted copolymers. Examples of a nonionic copolymer dispersant include polymeric compounds such as the tri-block EO-PO-EO co-polymers PLURONIC® L121, L123, L31, L81, L101 and P123 products. The term oligomer, as used herein, relates to a polymer compound consisting of only a few monomer units. Examples of ionic oligomer dispersants include SMA® 1440 and 2625 products.

The ionic or nonionic compound may also comprise a surfactant other than the surfactant compound of formula (1) of the present disclosure. Surfactants useful in the composition of the present disclosure are well known and include anionic, nonionic, cationic and amphoteric compounds. Combinations of more than one such compound may be used in the composition.

Anionic surfactant compounds which may be used include, but are not limited to, alkyl sulfates, alkyl benzene sulfonates, α-olefin sulfonates, alkyl taurates, alkyl sacrosinates, alkyl diphenyloxide disulfonates, alkyl naphthalene sulfonates, alkyl ether sulfates, alkyl ether sulfonates, sulfosuccinates, and other anionic surfactants as known for use in, for example, performance chemical formulations, including linear C₈-C₁₆ alkyl sulfates, C₈-C₁₆ alkyl sulfonates, C₈-C₁₆ alkyl benzene sulfonates and C₈-C₁₆ alkyl diphenyloxide disulfonates, decyl sulfophenoxy benzene/oxybis decyl benzene sulfonic acid disodium salt, and sodium octane sulfonate, sodium dodecyl sulfonate, sodium lauryl sulfate, and combinations of the foregoing. These surfactants are typically available as the alkali metal, alkaline earth and ammonium salts thereof.

Nonionic surfactant compounds which may be used include, but are not limited to, alkoxylates, N-substituted fatty acid amides, amine oxides, esters, sugar-based surfactants, polymeric surfactants, and mixtures thereof. Examples of alkoxylates are compounds such as non-aromatic alcohols, amines, amides, fatty acids or fatty acid esters which have been alkoxylated with 1 to 50 equivalents. Ethylene oxide and/or propylene oxide may be employed for the alkoxylation, usually ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters. Examples of polymeric surfactants are homo- or copolymers of vinylpyrrolidone, vinylalcohols, or vinylacetate.

Cationic surfactant compounds may also be used including quaternary surfactants, for example quaternary ammonium compounds with one or two hydrophobic groups, or salts of long-chain primary amines.

Amphoteric surfactant compounds which may be used include, but are not limited to, betaines, alkyl imidazolines, cocoamphopropionates, disodium cocoamphodipropionate (also known as cocoimidazoline carboxylate), or combinations thereof.

Other known additives, besides those described above, useful in performance chemical formulations and personal care formulations may optionally be added to the composition depending upon the application. These additives may include, but are not limited to, colorants, enzymes, wetting agents, antifoaming agents, buffering agents, pH adjusting agents, thickening agents, emulsifiers, anti-streaking agents, builders, chelating or sequestering agents, hydrotopes, anti-microbial agents, perfumes, herbicides, pesticides, fungicides, anti-oxidants, anti-wear additives, friction modifiers, viscosity index improvers, pour point depressants, corrosion inhibitors, solid carriers or fillers, protective colloids, adhesion agents, humectants, repellents, attractants, feeding stimulants, compatibilizers, bactericides, anti-freezing agents, crystallization inhibitors, tackifiers, binders, preservatives, clarifiers, fertilizers, UV stabilizers, salts, weighting agents, gravel particulates, gases, crosslinkers, thermodynamic hydrate inhibitors, kinetic hydrate inhibitors, clay stabilizing agents and mixtures thereof.

According to another aspect, there is provided a composition comprising the surfactant compound of formula (1) above and wherein the composition is substantially free of alkylphenol alkoxylates and polyarylphenol alkoxylates.

In yet another aspect, there is provided a packaged product comprising: a) a container having at least an outlet; and b) a composition comprising the surfactant compound of formula (1).

According to one aspect, the packaged product of the present disclosure comprises a container having a closure means, such as a lid, cover, cap, or plug to seal the container and/or at least one outlet. In another aspect, the sealed container also has a nozzle or pour spout. The sealed container may have the shape of a cylinder, oval, round, rectangle, canister, tub, square or jug and contains the composition comprising the surfactant compound of formula (1).

The container may be made from any material, such as steel, glass, aluminum, cardboard, tin-plate, plastics including, but not limited to, high density polyethylene (HDPE), polypropylene (PP), polyvinyl chloride (PVC), polyethylene terephthalate (PET), oriented polypropylene (OPP), polyethylene (PE) or polyamide and including mixtures, laminates or other combinations of these.

In another aspect, a concentrated composition comprising the surfactant compound of formula (1) is provided that may be further diluted in water and/or other solvents to form an aqueous solution. A concentrated composition of the present disclosure or “concentrate” allows one to dilute the concentrate to the desired strength and pH. A concentrate also permits longer shelf life and easier shipping and storage. Thus, in one aspect there is provided a concentrate containing the surfactant compound of the formula (1) of the present disclosure and water and/or solvent and optionally one or more additives described above. For the concentrate, the amount of water (and in some aspects, de-ionized water) and/or solvent may be, for instance, from about 0.5% by weight to about 50% by weight, based on the total weight of the concentrate. Accordingly, the amount of surfactant compound of formula (1) (and optional additives if present) contained in the concentrate may range from about 50% by weight up to 99.5% by weight, based on the total weight of the concentrate. As noted above, the concentrate may be further diluted with water, and in some embodiments, de-ionized water, and/or solvent to form the aqueous solution.

The composition including the surfactant compound of formula (1) of the present disclosure may be used in a variety of applications and compositions, including, but not limited to, performance chemical formulations and personal care formulations.

Thus, in one aspect there is provided a performance chemical formulation containing the composition comprising the surfactant compound of formula (1) wherein the surfactant compound of formula (1) is present in the performance chemical formulation in an amount ranging from about 0.01% by weight to about 40% by weight, based on the total weight of the performance chemical formulation. In another aspect, there is provided a performance chemical formulation containing the composition comprising the surfactant compound of formula (1) wherein the surfactant compound of formula (1) is present in the performance chemical formulation in an amount ranging from about 0.1% by weight to about 30% by weight, based on the total weight of the performance chemical formulation. In still another aspect, there is provided a performance chemical formulation containing the composition comprising the surfactant compound of formula (1) wherein the surfactant compound of formula (1) is present in the performance chemical formulation in an amount ranging from about 0.5% by weight to about 20% by weight, based on the total weight of the performance chemical formulation. In yet still another aspect, there is provided a performance chemical formulation containing the composition comprising the surfactant compound of formula (1) wherein the surfactant compound of formula (1) is present in the performance chemical formulation in an amount ranging from about 1% by weight to about 10% by weight, based on the total weight of the performance chemical formulation.

Accordingly, in still another aspect there is provided a personal care formulation containing the composition comprising the surfactant compound of formula (1) wherein the surfactant compound of formula (1) is present in the personal care formulation in an amount ranging from about 0.01% by weight to about 40% by weight, based on the total weight of the personal care formulation. In another aspect, there is provided a personal care formulation containing the composition comprising the surfactant compound of formula (1) wherein the surfactant compound of formula (1) is present in the personal care formulation in an amount ranging from about 0.1% by weight to about 30% by weight, based on the total weight of the personal care formulation.

In still another aspect, there is provided a personal care formulation containing the composition comprising the surfactant compound of formula (1) wherein the surfactant compound of formula (1) is present in the personal care formulation in an amount ranging from about 0.5% by weight to about 20% by weight, based on the total weight of the personal care formulation. In yet still another aspect, there is provided a personal care formulation containing the composition comprising the surfactant compound of formula (1) wherein the surfactant compound of formula (1) is present in the personal care formulation in an amount ranging from about 1% by weight to about 10% by weight, based on the total weight of the personal care formulation.

In one particular aspect, the performance chemical formulation is an agrochemical formulation. In other aspects, the agrochemical formulation may be an agrochemical emulsifiable concentrate or an agrochemical suspension concentrate.

In one particular aspect, there is provided an agrochemical emulsifiable concentrate containing an agrochemical active component, a solvent and the composition comprising the surfactant compound of formula (1).

Examples of agrochemical active components include, but are not limited to, a pesticide, fungicide, herbicide, insecticide, algicide, molluscicide, miticide, rodenticide, growth regulator or insect repellant. In one particular aspect, the agrochemical active component includes an insecticide, such as Malathion, Chlorpyrifos, Cypermethrin and Chloropicrin, a herbicide, such as Trifluralin, 2,4-D Ester, MCPA Isooctylester, Metolachlor, Acetochlor, Triclopyr and Roundup® weed and grass killer product, or a fungicide, such as Mefenoxam and Etridiazole. The agrochemical active component or components may be present in the agrochemical emulsifiable concentrate in an amount of at least about 5% w/w, or at least about 7.5% w/w, or at least about 10% w/w, where w/w means the weight of the agrochemical active component present in the agrochemical emulsifiable concentrate over the total weight of the agrochemical emulsifiable concentrate. In another aspect, the agrochemical active component or components may be present in the agrochemical emulsifiable concentrate in an amount of less than about 80% w/w, or less than about 75% w/w, or less than about 70% w/w, or less than about 65% w/w, or less than about 60% w/w, or less than about 55% w/w, or even less than about 50% w/w, where w/w means the weight of the agrochemical active component present in the agrochemical emulsifiable concentrate over the total weight of the agrochemical emulsifiable concentrate.

Examples of solvents include, but are not limited to, those described above. In another aspect, the solvent may be a hydrocarbon, ether, phenol, glycol, lactone, chlorinated hydrocarbon, aromatic hydrocarbon nitrated hydrocarbon, dibasic ester, mono-ester such as ethyl acetate, butyl acetate, ethyl-3-ethoxy-propionate, propylene glycol methyl ether acetate, propylene glycol butyl ether acetate, dipropylene glycol methyl ether acetate, dipropylene glycol butyl ether acetate and a cyclic ester such as butyrolactone, organic sulfur-containing compounds dimethylsulfoxide (DMSO) and sulfolane, methyl ethyl ketone (MEK), 5-methyl-2-hexanone (MIAK), methyl isobutyl ketone and methyl isoamylbutone, a glycol ether such as propylene glycol methyl ether (PM), dipropylene glycol methyl ether (DPM), or dipropylene glycol n-butyl ether (DPNB), ethylene glycol butyl ether (EB) and dipropylene glycol butyl ether (DB), an alcohol such as methanol, ethanol, propanol, butanol, benzyl alcohol, an amide and mixtures thereof. In some aspects, the solvent may be present in the agrochemical emulsifiable concentrate in an amount of at least 10% w/w, or at least about 15% w/w, or even at least about 20% w/w, where w/w means the weight of the solvent present in the agrochemical emulsifiable concentrate over the total weight of the agrochemical emulsifiable concentrate. In another aspect, the solvent may be present in the agrochemical emulsifiable concentrate in an amount of less than about 80% w/w, or less than about 75% w/w, or less than about 70% w/w, or less than about 65% w/w or even less than about 60% w/w, where w/w means the weight of the solvent present in the agrochemical emulsifiable concentrate over the total weight of the agrochemical emulsifiable concentrate

In another aspect, the composition comprising the surfactant compound of formula (1) may be present in the agrochemical emulsifiable concentrate in an amount of at least 1% w/w, or at least 2.5% w/w, or at least 5% w/w, or at least 7.5% w/w, where w/w means the weight of the composition comprising the surfactant compound of formula (1) present in the agrochemical emulsifiable concentrate over the total weight of the agrochemical emulsifiable concentrate. In another aspect, the composition comprising the surfactant compound of formula (1) may be present in the agrochemical emulsifiable concentrate in an amount of less than about 20% w/w, or less than about 15% w/w, or less than about 10% w/w, where w/w means the weight of the composition comprising the surfactant compound of formula (1) present in the agrochemical emulsifiable concentrate over the total weight of the agrochemical emulsifiable concentrate.

The agrochemical emulsifiable concentrate may optionally comprise one or more additional additives described above in an amount up to about 20% w/w, where w/w means the weight of the additives present in the agrochemical emulsifiable concentrate over the total weight of the agrochemical emulsifiable concentrate. In one aspect, the additives may be chosen from crystallization inhibitors, emulsifiers, surfactants other than the surfactant compound of formula (1), suspending agents, dyes, anti-oxidants, foaming agents, light absorbers, mixing aids, anti-foams, complexing agents, neutralizing or pH-modifying substances and buffers, corrosion-inhibitors, fragrances, wetting agents, absorption improvers, micronutrients, plasticizers, glidants, lubricants, dispersants, anti-freezes, and/or microbicides.

In another particular aspect, there is provided an agrochemical suspension concentrate comprising an agrochemical active component, water and the composition comprising the surfactant compound of formula (1).

In one aspect, the agrochemical suspension concentrate may comprise at least about 1% w/w, or at least about 2% w/w, or even at least about 5% w/w, of the agrochemical active component or components, where w/w means the weight of the agrochemical active component or components present in the agrochemical suspension concentrate over the total weight of the agrochemical suspension concentrate. In another aspect, the agrochemical suspension concentrate may comprise less than about 70% w/w, or less than about 65% w/w, or less than about 60% w/w, or less than about 55% w/w, or less than about 50% by weight, or less than about 45% w/w, or less than about 40% w/w of the agrochemical active component or components, where w/w means the weight of the agrochemical active component or components present in the agrochemical suspension concentrate over the total weight of the agrochemical suspension concentrate.

In another aspect, the agrochemical suspension concentrate may contain at least about 5% w/w of water, or at least about 10% w/w of water, or at least about 15% w/w of water, or at least about 20% w/w of water, or at least about 25% w/w of water, or at least about 30% w/w of water, or at least about 35% w/w of water, or even at least about 40% w/w, of water, where w/w means the weight of water present in the agrochemical suspension concentrate over the total weight of the agrochemical suspension concentrate. In another aspect, the agricultural suspension concentrate may comprise less than about 90% w/w of water, or less than about 80% w/w of water, or even less than about 75% w/w of water, where w/w means the weight of water present in the agrochemical suspension concentrate over the total weight of the agrochemical suspension concentrate.

In yet another aspect, the composition comprising the surfactant compound of formula (1) may be present in the agrochemical suspension concentrate in an amount of at least 0.5% w/w, or at least 1% w/w, or at least 2.5% w/w, where w/w means the weight of the composition comprising the surfactant compound of formula (1) present in the agrochemical suspension concentrate over the total weight of the agrochemical suspension concentrate. In another aspect, the composition comprising the surfactant compound of formula (1) may be present in the agrochemical suspension concentrate in an amount of less than about 10% w/w, or less than about 7.5% w/w, or less than about 5% w/w, where w/w means the weight of the composition comprising the surfactant compound of formula (1) present in the agrochemical suspension concentrate over the total weight of the agrochemical suspension concentrate.

The agrochemical suspension may optionally contain one or more additives described above in an amount of up to 20% w/w, where w/w means the weight of the additives present in the agrochemical suspension concentrate over the total weight of the agrochemical suspension concentrate.

### EXAMPLES

### Example 1. Synthesis by Alkoxylation

Diphenylmethanol was first reacted with a catalytic amount of potassium hydroxide and then reacted with propylene oxide (PO), ethylene oxide (EO), or a combination of both. The general reaction is shown below:

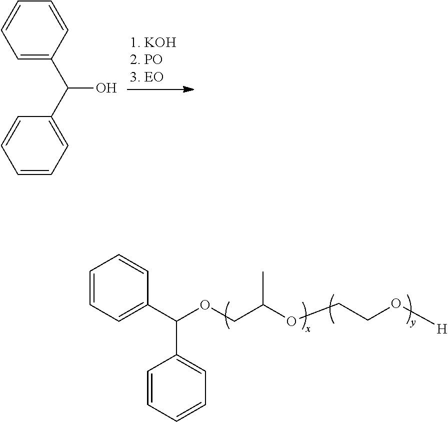

Table 1 provides various properties of the inventive surfactant compounds of the present disclosure derived from diphenylmethanol:

TABLE 1
Diphenylmethanol alkoxylate surfactant properties

| | | | Cloud point DI water | Draves | Equilibrium |
|---|---|---|---|---|---|
| Inventive | Moles | Moles | or | wetting | Surface |
| Compound | PO | EO | 10% NaCl (deg. C.) | time (sec) | Tension @ 0.1% |
| A | 0 | 5 | not soluble at RT | — | 48 |
| B | 0 | 7 | 40 | — | 44 |
| C | 0 | 10 | 72 | — | 51.5 |
| D | 0 | 12 | 85 | — | 52 |
| E | 0 | 14 | 90 | — | 52 |
| F | 4 | 12 | 76.2 | 76 | 39.3 |
| G | 4 | 17 | 91.5 | 68 | 41 |
| H | 4 | 25 | 69.5 (NaCl) | 540 | 42 |
| I | 6 | 6 | not soluble at RT | — | n/a |
| J* | 6 | 6 | 24 | 15 | 39 |
| K | 6 | 10 | 51 | 3 | 36.5 |
| L | 6 | 12 | 70 | 11 | 36 |
| M | 6 | 15 | 83.8 | 26 | 39 |
| N | 6 | 22 | 97.3 | 198 | 43 |
| O | 6 | 32 | 71 (NaCl) | 473 | 45 |
| P | 8 | 8 | 35 | 2 | 36 |
| Q | 8 | 10 | 51 | 3 | 36 |
| R | 8 | 12 | 59 | 5 | 35.5 |
| S | 8 | 15 | 73 | 3 | 35.6 |
| T | 10 | 8 | 23 | 2 | 35.7 |
| U | 10 | 10 | 45 | 3 | 35.7 |
| V | 10 | 12 | 55 | 2 | 35.5 |
| W | 10 | 15 | 67 | 4 | 35.4 |

Table 1 Notes:
Unless otherwise noted, alkoxylates were made by adding PO first, then EO.
*Made as a mixed alkoxide with EO and PO fed into the reactor simultaneously.

The surfactant compounds in Table 1 were synthesized by the following general procedure: 1 kg of diphenylmethanol (5.4 moles) was melted in a stainless steel reactor and a catalytic amount of 50% aqueous potassium hydroxide was then added. This mixture was heated to 120° C. under vacuum and water was removed. An appropriate amount of propylene oxide was then added and the reaction temperature was maintained at 125° C.-135° C. until the pressure equalized indicating that the propylene oxide had been consumed. An appropriate amount of ethylene oxide was then added at 150° C.-160° C. and the reaction was digested for 2 hours or until the pressure equalized. The resulting product was then neutralized with acetic acid and emptied from the reactor. The subsequently produced inventive surfactant compounds were then tested in an agrochemical formulations as emulsifiers. The first agrochemical formulation contained 70% acetochlor (a herbicide), 18% Aromatic 150 solvent (available from ExxonMobil Corp.), 6% NANSA® EVM 70/2E anionic surfactant (available from Innospec Performance Chemicals Europe Limited), and 6% of the diphenylmethanol alkoxylate surfactant compound. A control agrochemical formulation (not shown) that used NANSA® EVM 70/2E surfactant alone failed to form an emulsion.
Emulsions were then tested by adding 5 mL of the emulsifiable concentrate to 100 mL of soft, medium, or hard water in a graduated cylinder and placing them in a 25° C. water bath. Emulsion quality was evaluated over 4 hours. Table 2 shows the results of these tests.

TABLE 2

| | 34 ppm | | | 342 ppm | | | 1000 ppm | | |
|---|---|---|---|---|---|---|---|---|---|
| Compound | 30 min | 2 hr | 4 hr | 30 min | 2 hr | 4 hr | 30 min | 2 hr | 4 hr |
| B | 0 | 0 | tr | 3 ml oil | 5 ml oil | 5 ml oil | total separation | | |
| F | 0 | tr | tr | 0 | tr oil | tr oil | total separation | | |
| G | 1 ml | 3 ml | 3 ml | tr | 2 ml | 2 ml | tr | 1 ml | 2 ml |
| H | 2 ml | 3 ml | 4 ml | 2 ml | 3 ml | 4 ml | 2 ml | 2 ml | 3 ml |
| L | 0 | tr | 1 ml | 0 | 0 | 0 | 1 ml oil | total separation | |
| M | 1 ml | 2 ml | 3 ml | tr | 1 ml | 1 ml | 0 | tr | tr |
| N | 2 ml | 3 ml | 3 ml | 1 ml | 2 ml | 2 ml | 0 | 2 ml | 2 ml |
| O | 1 ml | 2 ml | 3 ml | 1 ml | 2 ml | 2 ml | 1 ml | 2 ml | 3 ml |

Table 2 notes:
#ml refers to how many ml of separation occurred
tr. = trace,
0 indicates no separation,
total separation means the organic and aqueous phases totally separated

Further optimization of the emulsifier blend using compound G resulted in even better performance. For this test, the agrochemical formulation included 70% acetochlor, 18% Aromatic 150 solvent, 7% NANSA® EVM 70/2E anionic surfactant and 5% of compound G. The results are provided in Table 3 below.

TABLE 3

| 34 ppm | | | | 342 ppm | | | | 1000 ppm | | | |
|---|---|---|---|---|---|---|---|---|---|---|---|
| 30 min | 1 hr | 2 hr | 4 hr | 30 min | 1 hr | 2 hr | 4 hr | 30 min | 1 hr | 2 hr | 4 hr |
| tr | 1 ml | 1 ml | 1 ml | 0 | 0 | 0 | 0 | 0 | 0 | 0 | 0 |

Another agrochemical EC formulation was tested which included 38% permethrin (an insecticide), 50% Aromatic 150 solvent, 6% NANSA® EVM 70/B (Innospec Performance Chemicals Europe Limited) surfactant, and 6% of the particular diphenylmethanol alkoxylate surfactant compound. Emulsions were tested by adding 5 mL of the emulsifiable concentrate to 100 mL of soft, medium, or hard water in a graduated cylinder and placing them in a 25° C. water bath. Emulsion quality was evaluated over 4 hours. The results for these tests are provided in Table 4 below.

TABLE 4

| | 34 ppm | | | 342 ppm | | | 1000 ppm | | |
|---|---|---|---|---|---|---|---|---|---|
| Com- | 30 | | | 30 | | | 30 | | |
| pound | min | 1 hr | 4 hr | min | 1 hr | 4 hr | min | 1 hr | 4 hr |
| F | tr | 1 ml | 4 ml | 0 | 0 | 1 ml | 0 | tr | 2 ml |
| G | 2 ml | 3 ml | 5 ml | 1 ml | 2 ml | 4 l | 1 ml | 2 ml | 4 ml |
| L | 0 | 0 | 0 | 0 | 0 | 0 | 0 | 0 | 0 |
| M | 0 | 0 | tr | 0 | 0 | 1 ml | 0 | 0 | 1 ml |

Table 4 notes:
#ml refers to how many ml of separation occurred
tr. = trace,
0 indicates no separation

As shown in the Tables above, the inventive surfactant compounds can be effectively used as nonionic emulsifiers to create stable agrochemical formulations.

### Interfacial Tension Reduction

The inventive compounds were also evaluated for their ability to reduce the interfacial tension between water and mineral oil. This is an important property that can indicate the effectiveness of a surfactant in cleaning and/or enhanced oil recovery applications. The interfacial tension between water containing 0.1% surfactant and mineral oil was measured using a drop volume tensiometer and compared to two standard surfactants as shown in FIG. 2 where LF-17 is SURFONIC® alcohol alkoxylate and L12-6 is SURFONIC® L12-6 alcohol alkoxylate (both available from Huntsman Petrochemical LLC). With no surfactant, the IFT was 55 mN/m.
As shown in FIG. 2, all surfactants can significantly reduce the IFT between water and oil. Additionally, the reduction of the interfacial tension with surfactant U of the inventive compounds is on par or better than one of the current industrial surfactants.

Example 2. Synthesis by Coupling with mPEGs

Diphenylmethanol was reacted with a methyl-terminated polyethylene glycol (mPEG, MW=350 g/mol) in the presence of an acid catalyst. The general reaction is shown below:

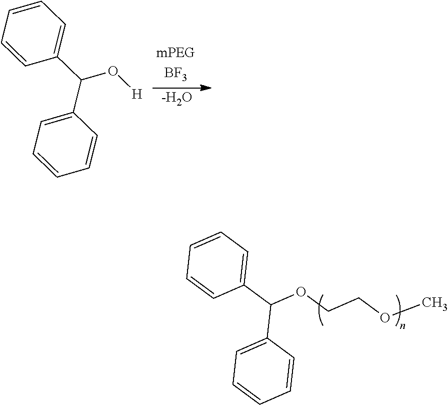

In this example, 200 grams of diphenylmethanol were mixed with an appropriate equimolar amount of mPEG by hydroxyl number in the presence of 0.2% (w/w) BF₃ (a Lewis acid) and then heated to 100° C. Methylcyclohexane (JEFFSOL® MCH product, available from Huntsman Petrochemical LLC) was used as an azeotropic solvent to assist with water removal. Water was collected in a dean-stark trap and the reaction was considered complete once water production ceased. After the reaction was complete, methycyclohexane was removed under vacuum at 80° C.
This method may also be carried out using polyethylene glycols, polyalkylene glycols or alcohol ethoxylates in place of the mPEG and adjusting the equivalents of diphenylmethanol accordingly.

### Example 3. Synthesis by Coupling with mPEGs

Triphenylmethanol was reacted with methyl-terminated polyethylene glycol (mPEG, MW=750 g/mol) in the presence of an acid catalyst. The general reaction is shown below:

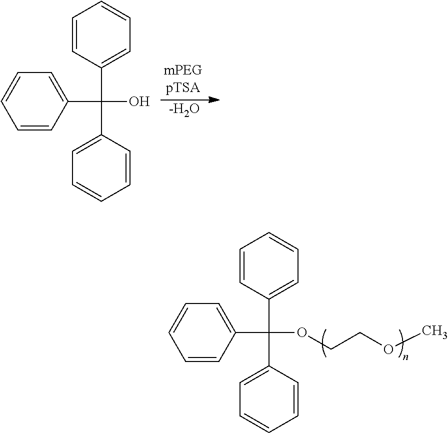

In this example, 200 grams of triphenylmethanol were mixed with an appropriate equimolar amount of a methyl-terminated polyethylene glycol (mPEG) determined by hydroxyl number in the presence of 0.2% para-toluenesulfonic acid (p-TSA) and then heated to 100° C. Methylcyclohexane was again used as an azeotropic solvent to assist with water removal. Water was collected in a dean-stark trap and the reaction was considered complete once water production ceased. The p-TSA was then neutralized with sodium bicarbonate. After the reaction was complete, the methycyclohexane was removed under vacuum at 80° C.

### Example 4. Synthesis of an Alkylated Benzhydrol and Subsequent Coupling with mPEG

The general reaction for the synthesis of alkylated benzhydrol is shown below:

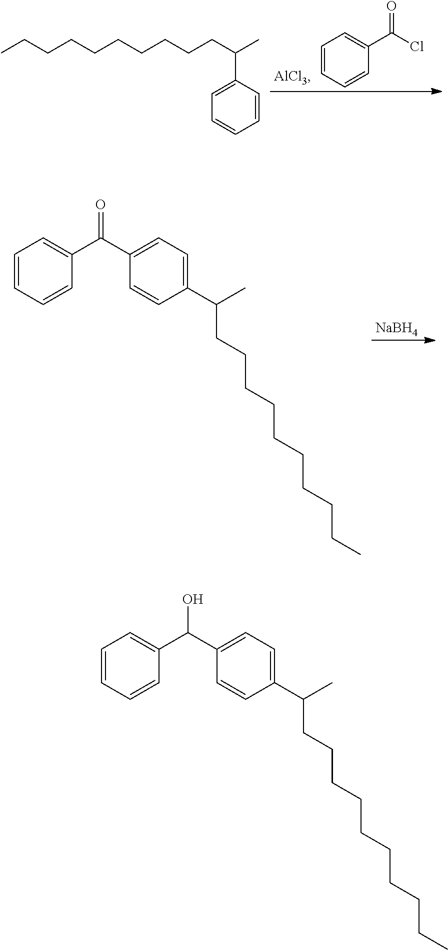

In this example, 101 grams of Alkylate® 225 linear alkyl benzene (a C₁₂ linear alkyl benzene) was reacted with 60 grams benzoyl chloride in the presence of 60 grams AlCl₃ catalyst in CH₂Cl₂ solvent at 0° C. for 2 hours. The temperature was then allowed to warm to room temperature and the mixture was stirred overnight. The reaction mixture was then poured over ice and worked up with aqueous K₂CO₃ and trisodium citrate, and the resulting organic layer was separated and washed three times with brine and dried over anhydrous sodium sulfate. The solvent was then removed under reduced pressure to yield a yellow oil, and the formation of the mono-alkyl acetophenone derivative was verified with GC and IR spectroscopy.
117 grams of the above alkylated derivative product was then dissolved in methanol and reacted with a stoichiometric amount of NaBH₄ to reduce the ketone to an alcohol. After solvent removal, a yellow oil was produced, and it was confirmed with IR spectroscopy to be the mono-alkyldiphenylmethanol product.
15.3 grams of the mono-alkyldiphenylmethanol product was then reacted at 150° C. in a melt with 33.1 grams of a methyl-terminated PEG-750 using p-TSA as a catalyst and vacuum was applied to strip off the water that had formed during the reaction. The resulting orange solid product was water-soluble and the static surface tension of a 0.1% solution of this material was 35 mN/m and it had a CMC value of 20 ppm, comparable with many commercial nonionic surfactants.

### Example 5—Sulfation of Diphenylmethyl Alkoxylates

Extended chain surfactants can be used to enhance the solubilization of certain oils (see, for e.g. U.S. Pat. Nos. 7,467,633 and 8,172,953) and give improved cleaning or oil emulsification properties to formulations. An extended chain ether sulfate was made by reacting diphenylmethanol with 10 moles of PO, followed by 3 moles of EO, according to the procedure in Example 1. Then, 100 grams of the material was sulfated using an appropriate amount of sulfamic acid and a high shear mixer at 100° C., according to the scheme below:

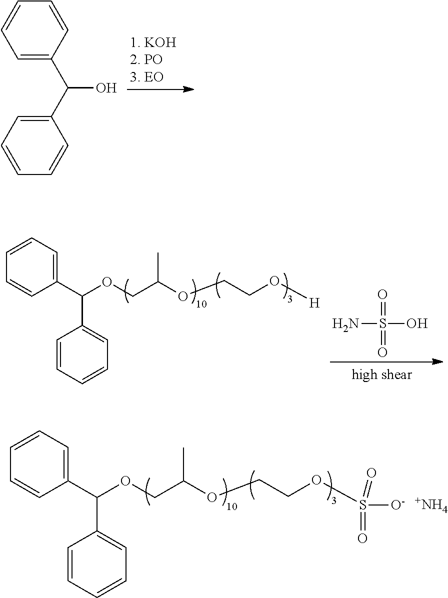

### Example 6—Phosphate Esters

In this example, 100 grams of the compound shown below was reacted with 10 grams of polyphosphoric acid using a high shear mixer. The temperature was maintained at 100° C. for 4 hours to produce the product, according to the scheme below:

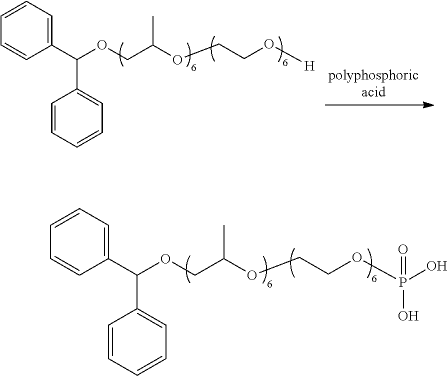

### Example 7 Detergency

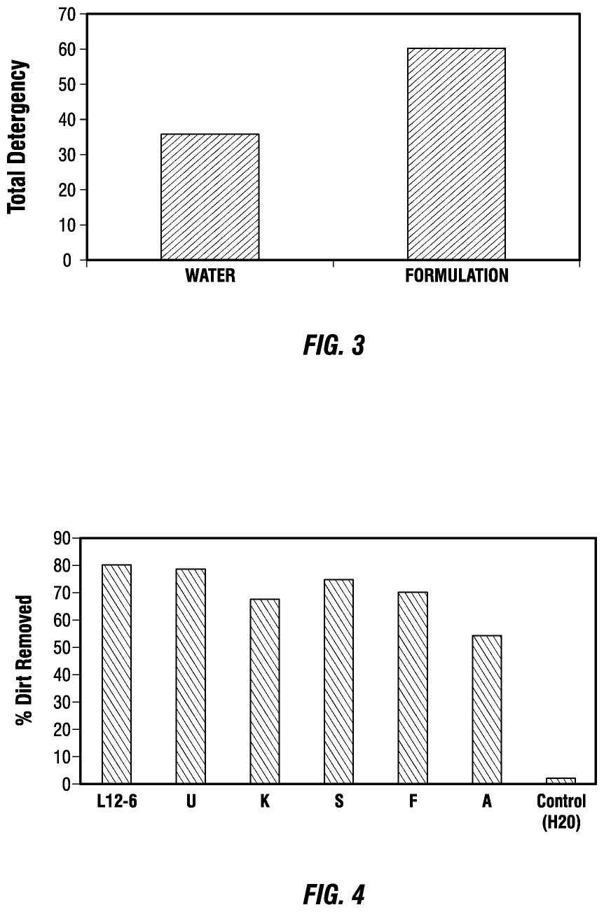

To study the performance of the inventive surfactant compounds in detergent formulations, they were used as the nonionic portion of a standard liquid laundry detergent mix, as shown in Table 5. An in-house method for measuring detergency was developed which utilizes a Tergotometer, soiled cloth swatches and a spectrophotomer. To begin, various soiled swatches obtained from Test Fabrics, were read with a HunterLab spectrophotometer.
Next, the soiled swatches were added to the Tergotometer along with 200 ppm of Laundry Detergent Formulation A and 1L 150 ppm hard water. A 15 minute wash cycle at 100 RPM and 37.78° C. was followed by a 5 minute rinse cycle at 100 RPM and 37.78° C. The Tergotometer of choice has six 1-L temperature controlled buckets with mechanical agitation to simulate an actual washing machine. Testing was always carried out with at least one control and water as a baseline. Following the rinse cycle, the swatches were placed flat on a mesh screen in an oven at 65° C. to dry. After the swatches were dried, they were read again on the spectrophotometer to measure the amount of soil that was removed. The results are shown in FIG. 3.

TABLE 5
Detergency Formulation A

| Component | wt. % |
|---|---|
| Water | 61.349 |
| Compound L | 25.55 |
| Triethanolamine | 2.8 |
| Alkylbenzenesulfonic acid | 5.25 |
| Acusol 445N | 0.5 |
| Glycerin | 4 |
| NaCl | 0.25 |
| Optical brightener | 0.1 |
| Preservative | 0.1 |
| Dye | 0.001 |
| Fragrance | 0.1 |

The results depicted in FIG. 3 show that the formulation cleaned the swatches better than water alone.

### Example 8: Hard Surface Cleaning

Another important application of surfactants includes their addition as components in hard surface cleaning formulations. The inventive compounds were used as the nonionic surfactant portion of a standard hard surface cleaning formulation and evaluated using an abrasion tester to scrub tiles coated with a standard soil consisting of carbon black, bentonite clay, and vegetable grease. The formulation ingredients are outlined in Table 6 and the performance of the formulations is shown in FIG. 4.

TABLE 6

| Component | wt. % |
|---|---|
| Water | 87.4 |
| tetrapotassium | 1.5 |
| pyrophosphate | |
| sodium citrate | 1.1 |
| Nonionic surfactant | 5 |
| Ethylene glycol butyl ether | 5 |

Formulations containing the inventive surfactants all performed better than the control, and some were comparable in performance to an industry standard nonionic surfactant, SURFON IC® L12-6 alkoxylate (available from Huntsman Petrochemical LLC).

While the foregoing is directed to various aspects of the present disclosure, other and further aspects of the disclosure may be devised without departing from the basic scope thereof, and the scope thereof is determined by the claims that follow.

1. A composition comprising a surfactant compound having the formula (1) 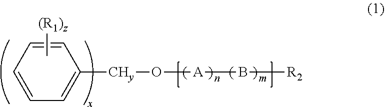
where R₁ is independently hydrogen or a C₁-C₁₈ alkyl group; A is ethyleneoxy, B is a C₃ alkyleneoxy, a C₄ alkyleneoxy or a mixture thereof, where A and B may be randomly distributed, or in the form of two more blocks of any order, R₂ is hydrogen, a C₁-C₁₆ alkyl group, benzyl, diphenylmethyl, triphenylmethyl, SO₃M, CH₂COOM or PO₃M; M is hydrogen, a water-soluble cation, a monovalent metal or a polyvalent metal cation; m is an integer from 0 to 60; n is an integer from 0 to 60; x is 2 or 3; y is 0 or 1; each z is 0, 1 or 2; and wherein m+n=4 to 100 and x+y=3.

2. The composition of claim 1, wherein x is 2, y is 1 and each z is 0.

3. The composition of claim 1, wherein x is 2, y is 1, at least one z is 1 and R₁ is a C₄-C₁₄ alkyl group when z is 1.

4. The composition of claim 1, wherein x is 3 and each z is 0.

5. The composition of claim 1, wherein x is 3, y is 1, at least one z is 1 and R₁ is a C₄-C₁₄ alkyl group when z is 1.

6. The composition of claim 1, wherein R₂ is hydrogen, methyl, diphenylmethyl, triphenylmethyl or a C₄-C₁₆ alkyl.

7. The composition of claim 1, wherein m is an integer of 0 to 25, n is an integer from 0 to 25, and m+n=10 to 30.

8. A method of preparing a surfactant compound having the formula (1) 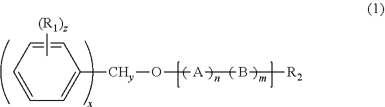
where R₁ is independently hydrogen or a C₁-C₁₈ alkyl group; A is ethyleneoxy, B is a C₃ alkyleneoxy, a C₄ alkyleneoxy or a mixture thereof, where A and B may be randomly distributed, or in the form of two more blocks of any order, R₂ is hydrogen, a C₁-C₁₆ alkyl group, benzyl, diphenylmethyl, triphenylmethyl, SO₃M, CH₂COOM or PO₃M; M is hydrogen, a water-soluble cation, a monovalent metal or a polyvalent metal cation; m is an integer from 0 to 60; n is an integer from 0 to 60; x is 2 or 3; y is 0 or 1; each z is 0, 1 or 2; and wherein m+n=4 to 100 and x+y=3 comprising the steps of direct alkoxylation of diphenylmethanol, triphenylmethanol or an alkylated derivative of diphenylmethanol or triphenylmethanol with ethylene oxide, propylene oxide, butylene oxide or a mixture thereof and optionally further reaction with an acidic moiety and optionally neutralization with a source of alkali metal, alkaline earth metal, amine or ammonia to form the surfactant compound of formula (1).

9. A method of preparing a surfactant compound having the formula (1) 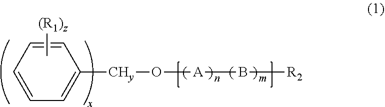
where R₁ is independently hydrogen or a C₁-C₁₈ alkyl group; A is ethyleneoxy, B is a C₃ alkyleneoxy, a C₄ alkyleneoxy or a mixture thereof, where A and B may be randomly distributed, or in the form of two more blocks of any order, R₂ is hydrogen, a C₁-C₁₆ alkyl group, benzyl, diphenylmethyl, triphenylmethyl, SO₃M, CH₂COOM or PO₃M; M is hydrogen, a water-soluble cation, a monovalent metal or a polyvalent metal cation; m is an integer from 0 to 60; n is an integer from 0 to 60; x is 2 or 3; y is 0 or 1; each z is 0, 1 or 2; and wherein m+n=4 to 100 and x+y=3 comprising the steps of direct condensation of diphenylmethanol, triphenylmethanol or an alkylated derivative of diphenylmethanol or triphenylmethanol with a hydroxyl-terminated polyglycol ether in the presence of an acid catalyst and optionally further reaction with an acidic moiety and optionally neutralization with a source of alkali metal, alkaline earth metal, amine or ammonia to form the surfactant compound of formula (1).

10. A packaged product comprising: a) a container having at least an outlet; and b) the composition of claim 1.

11. A performance chemical formulation comprising the composition of claim 1, wherein the surfactant compound of formula (1) is present in the performance chemical formulation in an amount ranging from about 0.01% by weight to about 40% by weight, based on the total weight of the performance chemical formulation.

12. The performance chemical formulation of claim 11, wherein the surfactant compound of formula (1) is present in the performance chemical formulation in an amount ranging from about 1% by weight to about 10% by weight, based on the total weight of the performance chemical formulation.

13. A personal care formulation comprising the composition of claim 1, wherein the surfactant compound of formula (1) is present in the personal care formulation in an amount ranging from about 0.01% by weight to about 40% by weight, based on the total weight of the personal care formulation.

14. The personal care formulation of claim 13, wherein the surfactant compound of formula (1) is present in the personal care formulation in an amount ranging from about 1% by weight to about 10% by weight, based on the total weight of the personal care formulation.

15. An agrochemical emulsifiable concentrate comprising an agrochemical active component, a solvent and the composition of claim 1.

16. An agrochemical suspension concentrate comprising an agrochemical active component, water and the composition of claim 1.
